(19) United States
(12) Patent Application Publication
(10) Pub. No.: US 2012/0065177 A1
(43) Pub. Date: Mar. 15, 2012

(54) PHARMACEUTICAL FORMULATIONS COMPRISING AZELASTINE AND A CORTICOSTEROID FOR THE TREATMENT OF INFLAMMATORY OR ALLERGIC CONDITIONS

(75) Inventors: David Douglas Myles, Stevenage (GB); David Hugh Richards, Greenford (GB)

(73) Assignee: Glaxo Group Limited, Greenford (GB)

(21) Appl. No.: 13/204,978

(22) Filed: Aug. 8, 2011

Related U.S. Application Data
(62) Division of application No. 12/374,523, filed on Jan. 21, 2009.

(30) Foreign Application Priority Data
Jul. 28, 2006 (GB) 0615108.8

(51) Int. Cl. A61K 31/58; A61P 11/02

(52) U.S. Cl. 514/172

(57) ABSTRACT

The present invention relates to method for the treatment of allergic rhinitis comprising: administrating to a patient in need thereof a pharmaceutical formulation comprising a compound for formula (I)

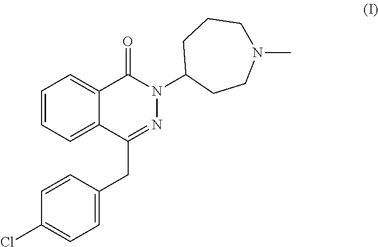

or a salt thereof, and an anti-inflammatory glucocorticoid compound of formula (II)

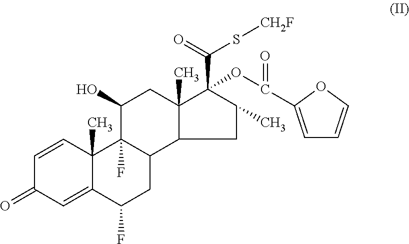

or a solvate thereof.

### CROSS-REFERENCE TO RELATED APPLICATIONS

This application is a divisional application of U.S. patent application Ser. No. 12/374,523 filed Jan. 21, 2009, which is a 35 U.S.C. §371 United States National Phase Application of International Patent Application No. PCT/EP2007/057695 filed Jul. 26, 2007, which claims priority from Great Britain Patent Application No. 0615108.8 filed in the United Kingdom on Jul. 28, 2006.

### FIELD OF THE INVENTION

The present invention relates to pharmaceutical formulations containing an anti-inflammatory glucocorticoid compound of the androstane series and azelastine, an H₁ antagonist/anti-allergic. The present invention also relates to therapeutic uses thereof, particularly for the treatment of inflammatory and allergic conditions, specifically rhinitis.

### BACKGROUND OF THE INVENTION

Glucocorticoids which have anti-inflammatory properties are known and are widely used for the treatment of inflammatory disorders or diseases such as asthma and rhinitis. For example, U.S. Pat. No. 4,335,121 discloses 6α,9α-difluoro-17α-(1-oxopropoxy)-1,0-hydroxy-16α-methyl-3-oxo-androsta-1,4-diene-17β-carbothioic acid S-fluoromethyl ester (known by the generic name of fluticasone propionate) and derivatives thereof. The use of glucocorticoids generally, and especially in children, has been limited in some quarters by concerns over potential side effects. The side effects that are feared with glucocorticoids include suppression of the Hypothalamic-Pituitary-Adrenal (HPA) axis, effects on bone growth in children and on bone density in the elderly, ocular complications (cataract formation and glaucoma) and skin atrophy. Certain glucocorticoid compounds also have complex paths of metabolism wherein the production of active metabolites may make the pharmacodynamics and pharmacokinetics of such compounds difficult to understand. Whilst the modern glucocorticoids are very much safer than those originally introduced, it remains an object of research to produce new molecules and formulations of old and new molecules which have excellent anti-inflammatory properties, with predictable pharmacokinetic and pharmacodynamic properties, with an attractive side effect profile, and with a convenient treatment regime.

In the following applications we have identified novel glucocorticoid compounds which substantially meet these objectives, WO02/12265, WO02/12266, WO05/005452, WO05/005451 and WO02/088167.

H₁ antagonists/antiallergics are known and can be used in nasal sprays and eye drops to treat allergy-related conditions. It is known that azelastine, an H₁ antagonist/antiallergic (usually as the hydrochloride salt) for example as disclosed in U.S. Pat. No. 3,813,384, can be administered as a nasal spray to treat such conditions, for example rhinitis.

Formulations comprising azelastine or a pharmaceutically acceptable salt, solvate or physiologically functional derivative thereof and a steroid or a pharmaceutically acceptable salt, solvate or physiologically functional derivative thereof, have been disclosed, for example, in WO03/105856.

We have now identified formulations comprising particular corticosteroidal compounds or solvates thereof and azelastine, these formulations are suitable for intranasal administration and may have advantages over those formulations already known.

Many millions of individuals suffer from seasonal and perennial allergic rhinitis worldwide. Symptoms of seasonal and perennial allergic rhinitis include nasal itch, congestion, runny nose, sneezing and watery eyes. Seasonal allergic rhinitis is commonly known as ‘hay fever’. It is caused by allergens which are present in the air at specific times of the year, for example tree pollen during Spring and Summer. Perennial allergic rhinitis is caused by allergens which are present in the environment during the entire year, for example dust mites, mould, mildew and pet dander.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the greater the efficacy, and therefore the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10 to 30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors. In the case of administration of glucocorticoids, the potential for any undesirable side effects should preferably be minimised.

### SUMMARY OF THE INVENTION

Thus, according to one aspect of the invention, there is provided a pharmaceutical formulation comprising a compound of formula (I)

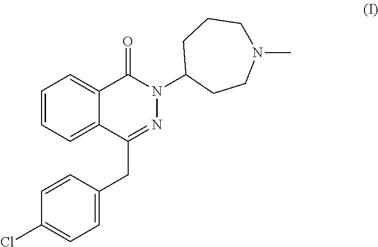

or a salt or solvate thereof
and a corticosteroid of formula (II)

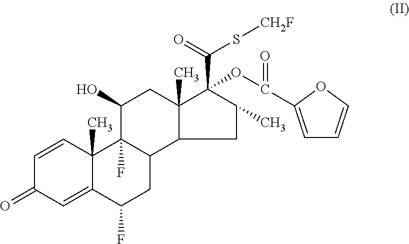

or a solvate thereof.

According to another aspect of the invention, there is provided a pharmaceutical formulation comprising a compound of formula (I)

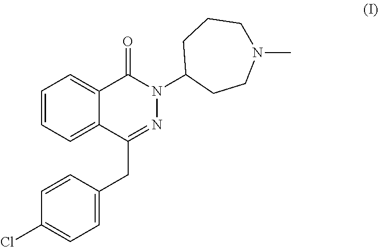

or a salt or solvate thereof
and a corticosteroid of formula (III)

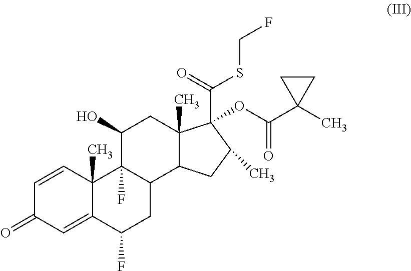

or a solvate thereof.

According to another aspect of the invention, there is provided, a pharmaceutical formulation comprising a compound of formula (I)

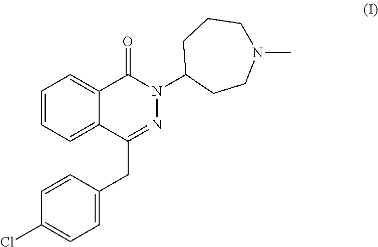

or a salt or solvate thereof
and a corticosteroid of formula (IV)

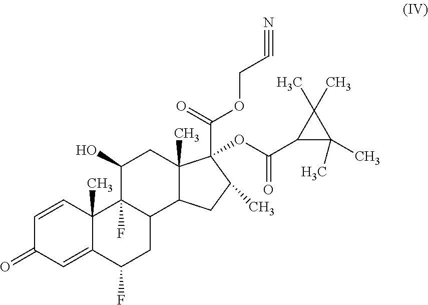

or a solvate thereof.

According to another aspect of the present invention, there is provided a pharmaceutical formulation comprising a compound of formula (I)

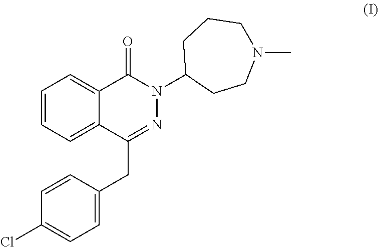

or a salt or solvate thereof
and a corticosteroid of formula (V)

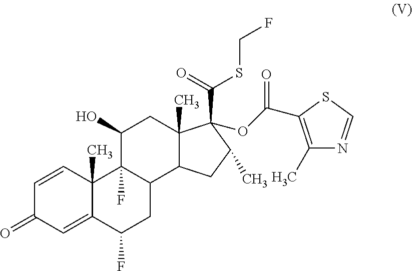

or a solvate thereof.

The advantages of the formulations of the present invention may include that the formulations demonstrate good anti-inflammatory properties, good antiallergic properties, with an attractive side-effect profile, rapid onset of action, long duration of action, and compatibility with a convenient regime of treatment in human patients, and may be amendable to once-per day dosing and improved efficacy. In addition the combination may allow lower does of one or both of the components to be used leading to an improved safety profile. Further advantages may include the fact that the formulation has desirable physical and chemical properties which allow for ready manufacture and storage.

In one embodiment the compound of formula (II) is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

In another embodiment the compound of formula (III) is 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester.

In another embodiment the compound of formula (IV) is 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester.

In another embodiment the compound of formula (V) is 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester.

In one embodiment the compound of formula (I) is azelastine, either in racemic form or as a single enantiomer.

In some embodiments the azelastine is present in the formulation as azelastine hydrochloride.

In some aspects of the invention there is provided a pharmaceutical formulation wherein the corticosteroid is present in the form of suspended particles and the azelastine is present in dissolved form.

In some aspects of the invention there is provided a pharmaceutical formulation which is an aqueous pharmaceutical formulation.

In some aspects of the invention there is provided a pharmaceutical formulation suitable for intranasal delivery.

In some embodiments the formulation will contain one or more suspending agents.

In some embodiments the formulation will contain one or more preservatives.

In some embodiments the formulation will contain one or more wetting agents.

In some embodiments the formulation will contain one or more isotonicity adjusting agents.

In some embodiments the formulation will contain a buffer.

In some embodiments the formulation will contain one or more taste-masking agents.

According to one aspect of the present invention we provide a pharmaceutical formulation which comprises:

(i) an aqueous solution of a compound of formula (I) or a salt or solvate thereof;
(ii) an aqueous suspension of a corticosteroid selected from the group consisting of a compound of formula (II), a compound of formula (III), a compound of formula (IV) and a compound of formula (V);
(iii) one or more suspending agents;
(iv) one or more preservatives;
(v) one or more wetting agents;
(vi) a buffer;
(vii) one or more isotonicity adjusting agents; and optionally
(viii) one or more taste-masking agents.

In another aspect of the invention there is provided a pharmaceutical formulation which is free of preservative.

The formulations of the present invention may be stabilised by the appropriate selection of pH. Typically, the pH will be adjusted to 3.0 to 8.0, in one embodiment 4.0 to 7.0, for example around 4.5, to maximise the efficacy of the preservative.

Examples of pharmaceutically acceptable materials which can be used to adjust the pH of the formulation include hydrochloric acid and/or sodium hydroxide. In one embodiment, the pH of the formulation will be adjusted using hydrochloric acid.

It is also possible to add to the formulations buffer substances such as citric acid/sodium hydrogensulphate borate buffer, citric acid/citrate buffer, phosphates (sodium hydrogenorthophosphate, disodium hydrogenphosphate), trometamol or equivalent conventional buffers in order to adjust the pH value of the formulation. In one embodiment the buffer comprises a citric acid/citrate buffer, for example a citric acid/sodium citrate buffer.

In one embodiment the amount of citric acid is from 0.1 to 1.5 g, for example from 0.5 to 1.0 g and the amount of sodium citrate is from 0.5 to 2.0 g, for example from 1.0 to 2.0 g per 100 ml of solution. The weights given relate in each case to the anhydrous substances.

The aqueous component is desirably a high grade quality of water, for example purified water.

The active compound of formula (II), (III), (IV), (V) or solvate thereof will suitably have a mass mean diameter (MMD) of less than 20 μm, in one embodiment between 0.5-10 μm, for example between 1-5 μm. If particle size reduction is necessary, this may be achieved by techniques such as micronisation and/or microfluidisation.

In one embodiment the MMDs are between 2-4 μm.

In some embodiments, if necessary, particle size reduction may be achieved by micronisation.

In other embodiments, particle size reduction may be achieved by microfluidisation.

In one embodiment, the particles will be crystalline, prepared for example by a process which comprises mixing in a continuous flow cell in the presence of ultrasonic radiation a flowing solution of compound of formula (II), (III), (IV), (V) or solvate thereof as medicament in a liquid solvent with a flowing liquid antisolvent for said medicament (for example, as described in WO00/38811).

In the pharmaceutical formulations of the invention, the compounds of formula (II), (III), (IV), (V) or solvate thereof may be present within the formulation in an amount of from 0.005% to 1% (w/w), in one embodiment from 0.01% to 0.5% (w/w), for example from 0.05 to 0.1% (w/w) based on the total weight of the formulation. Typically, 50 μl of suspension will contain 50 μg of compound of formula (II), (III), (IV), (V) or solvate thereof.

In the pharmaceutical formulations of the invention, the compound of formula (I), or a salt or solvate thereof, may be present within the formulation in an amount of from 0.0005% to 2% (w/w), in one embodiment from 0.01% to 0.6% (w/w), for example from 0.1 to 0.3% (w/w) based on the total weight of the formulation.

Examples of suspending agents include cellulose, carboxymethylcellulose, veegum, tragacanth, bentonite, methylcellulose and polyethylene glycols. In one embodiment the suspending agent will be microcrystalline cellulose and carboxy methylcellulose sodium, for example used as the branded product Avicel RC591 (which typically contains 87-91% microcrystalline cellulose and 9-13% carboxy methylcellulose sodium) or Avicel CL611. In one embodiment particulate microcrystalline cellulose has a particle size in the range 1 to 100 μm. We believe that Avicel RC591 acts as a suspending agent by imparting thixotropic properties to the formulation, wherein the formulation may become a stable suspension upon being stirred, shaken or otherwise disturbed.

In some embodiments, the thixotropic nature of the suspending agent will ensure that the formulation assumes a gel like appearance at rest, wherein the particulate medicament is dispersed and suspended substantially uniformly, characterised by a high viscosity value. Once the composition is subjected to shear forces, such as those caused by agitation prior to spraying, the viscosity of the formulation will decrease to such a level to enable it to flow readily through the spray device and exit as a spray of fine particles in a mist. These particles will then be capable of infiltrating the mucosal surfaces of the anterior regions of the nose (frontal nasal cavities), the frontal sinus, the maxillary sinuses and the turbinates which overlie the conchas of the nasal cavities. Once deposited, the viscosity of the formulation will increase to a sufficient level to assume its gel-like form and resist being cleared from the nasal passages by the inherent mucocillary forces that are present in the nasal cavities.

When the formulation of the present invention comprises a suspending agent, it will desirably be added in a suitable amount to achieve this function. In some embodiments the suspending agent will be present within the formulation in an amount of from 0.1 to 5% (w/w), for example 1.5% (w/w), based on the total weight of the formulation.

For stability purposes, the formulation of the present invention may be protected from microbial contamination and growth by inclusion of a preservative. Examples of pharmaceutically acceptable anti-microbial agents or preservatives that can be used in the formulation include quaternary ammonium compounds (for example benzalkonium chloride, benzethonium chloride, cetrimide, cetylpyridinium chloride and myristyl picolinium chloride), alcoholic agents (for example chlorobutanol, phenylethyl alcohol and benzyl alcohol), antibacterial esters (for example esters of para-hydroxybenzoic acid), chelating agents such as disodium edetate (EDTA), and other anti-microbial agents such as chlorhexidine (for example in the form of the acetate or gluconate), potassium sorbate, chlorocresol, sorbic acid and its salts, polymyxin, methylparaben and propylparaben.

In some embodiments the preservative may comprise disodium edetate (EDTA), which may be present within the formulation in an amount of from 0.001 to 1% (w/w), for example around 0.015% (w/w), based on the total weight of the formulation.

In some embodiments the preservative may comprise benzalkonium chloride (BKC), which may be present within the formulation in an amount of from 0.001 to 1% (w/w), for example around 0.015% (w/w), based on the total weight of the formulation.

In some embodiments, the preservative may comprise disodium edetate and benzalkonium chloride or disodium edetate and potassium sorbate, in one embodiment potassium chloride and/or disodium edetate.

Formulations, for example nasal formulations which contain a suspended medicament (such as a compound of formula (II), (III), (IV), (V) or a solvate thereof) may contain a pharmaceutically acceptable wetting agent which functions to wet the particles of medicament to facilitate dispersion thereof in the aqueous phase of the composition. It is desirable that the amount of wetting agent used will not cause foaming of the dispersion during mixing.

It will be appreciated that any agent which is effective in wetting the particles and which is pharmaceutically acceptable can be used. Examples of wetting agents that can be used are fatty alcohols, esters and ethers. In one embodiment the wetting agent is a hydrophilic, non-ionic surfactant, for example polyoxyethylene (20) sorbitan monooleate (supplied as the branded product Polysorbate 80).

Wherein the formulation of the present invention comprises a wetting agent, it will desirably be added in a sufficient quantity to achieve this function. In one embodiment the wetting agent may be present within the formulation in an amount of from 0.001 to 0.05% (w/w), for example 0.025% (w/w), based on the total weight of the formulation.

The presence of an isotonicity adjusting agent is to achieve isotonicity with body fluids, for example fluids of the nasal cavity, resulting in reduced levels of irritancy associated with many nasal formulations. Examples of suitable isotonicity adjusting agents are glucose, glycerine, sorbitol, sodium chloride, dextrose and calcium chloride. In one embodiment the isotonicity adjusting agent may be dextrose, for example, anhydrous dextrose.

When the formulation of the present invention comprises an isotonicity adjusting agent it will be desirably added in a sufficient quantity to achieve this function, in one embodiment the isotonicity adjusting agent will be present within the formulation in an amount of from 0.1 to 10% (w/w), for example 5.0% w/w, based on the total weight of the formulation.

Further auxiliary substances which may, for example, be used for the formulations of the invention are: polyvinyl pyrrolidone, sorbitan fatty acid esters such as sorbitan trioleate, polyethoxylated sorbitan fatty acid ester (for example polyethoxylated sorbitan trioleate), sorbimacrogol oleate, synthetic amphotensides (tritons), ethylene oxide ethers of octylphenolformaldehyde condensation products, phosphatides such as lecithin, polyethoxylated fats, polythoxylated oleotriglycerides and polyethoxylated fatty alcohols. In this context polyethoxylated means that the relevant substances contain polyoxyethylene chains, the degree of polymerisation of which is generally between 2 to 40, in particular between 10 to 20. These substances are generally used to improve the solubility of the azelastine component.

The formulations of the present invention may also contain further excipients and/or carriers that reduce the amount of post-nasal drip, and/or minimise or mask the unpleasant bitter taste associated with post-nasal drip of formulations comprising azelastine, for example those disclosed in US application US 2006/0110331.

Examples of taste-masking agents include sucralose, sucrose, saccharin or a salt thereof, fructose, dextrose, corn syrup, aspartame, acesulfame-K, xylitol, sorbitol, erythritol, ammonium glycyrrhizinate, thaumatin, neotame, mannitol, menthol, eucalyptus oil, camphor, a natural flavouring agent, an artificial flavouring agent, and combinations thereof. In one embodiment the taste-masking agent is sucralose and/or menthol.

The pharmaceutical formulation according to the invention may further comprise one or more excipients. By the term “excipient”, as used herein, it is meant to mean substantially inert materials that are nontoxic and do not interact with other components of a composition in a deleterious manner including, but not limited to, pharmaceutical grades of: carbohydrates, organic and inorganic salts, polymers, amino acids, phospholipids, wetting agents, emulsifiers, surfactants, poloxamers, pluronics, and ion exchange resins, and combinations thereof, a non-exhaustive list of examples of which are provided below:

Carbohydrates, including: monosaccharides, such as, but not limited to, fructose; disaccharides, such as, but not limited to lactose, and combinations and derivatives thereof; polysaccharides, such as, but not limited to, cellulose and combinations and derivatives thereof; oligosaccharides, such as, but not limited to, dextrins, and combinations and derivatives thereof; polyols, such as but not limited to sorbitol, and combinations and derivatives thereof;

Organic and inorganic salts, including but not limited to sodium or calcium phosphates, magnesium stearate, and combinations and derivatives thereof;

Polymers, including: natural biodegradable protein polymers including, but not limited to, gelatin and combinations and derivatives thereof;

Natural biodegradable polysaccharide polymers including, but not limited to, chitin and starch, crosslinked starch and combinations and derivatives thereof;

Semisynthetic biodegradable polymers including, but not limited to, derivatives of chitosan;

Synthetic biodegradable polymers including but not limited to polyethylene glycols (PEG), polylactic acid (PLA), synthetic polymers including but not limited to polyvinyl alcohol and combinations and derivatives thereof;

Amino acids including but not limited to including non-polar amino acids, such as leucine and combinations and derivatives thereof;

Phospholipids, including lecithins and combinations and derivatives thereof;

Wetting agents/Surfactants/Emulsifiers, including, but not limited to gum acacia, cholesterol, fatty acids including, combinations and derivatives thereof;

Poloxamers/Pluronics: including but not limited to poloxamer 188, Pluronic® F-108, and combinations and derivations thereof;

Ion exchange resins: including but not limited to amberlite IR120 and combinations and derivatives thereof;

and combinations of the noted excipients.

In the pharmaceutical formulation of the invention, in one embodiment the suspending agent is microcrystalline cellulose and carboxy methylcellulose sodium, the preservative is EDTA and potassium sorbate, the wetting agent is polyoxyethylene (20) sorbitan monooleate and the isotonicity adjusting agent is dextrose and/or glucose.

Preferable means for applying the formulation of the present invention to the nasal passages is by use of a pre-compression pump, such as a VP3, VP7 or modifications, model manufactured by Valois SA. Advantages of pumps of this type are beneficial as they will ensure that the formulation is not released or atomised until a sufficient force has been applied, otherwise smaller doses may be applied. Typically, these pre-compression pumps may be used with a bottle (glass or plastic) capable of holding 8-50 ml of a formulation. Each spray will typically deliver 50-100 μl of such a formulation, therefore, the device is capable of providing at least 100 metered doses. Suitably the formulation will be dispensed from a vessel fitted with a suitable pre-compression pump and nasal actuator, adapted to dispense 50 or 100 μl per actuation, preferably 50 μl. There is therefore provided a device adapted for intranasal delivery of a pharmaceutical formulation comprising a pharmaceutical formulation of the present invention.

A suitable dosing regime for the formulation of the present invention when administered to the nose would be for the patient to inhale slowly through the nose subsequent to the nasal cavity being cleared. During inhalation the formulation would be applied to one nostril while the other is manually compressed. This procedure would then be repeated for the other nostril.

Typically, one or two inhalations per nostril would be administered by the above procedure up to three times each day, possibly twice daily, ideally once daily.

It will be appreciated that the above dosing regime should be adjusted according to the patient's age, body weight and/or symptom severity.

The formulations of the present invention have potentially beneficial anti-inflammatory or anti-allergic effects, particularly upon topical administration to the nose. Hence, formulations according to the invention are useful in the treatment of inflammatory and/or allergic disorders of the nose, especially in once-per-day therapy.

Formulations according to the invention may be prepared by combining the ingredients in water. If necessary the pH may be adjusted as a final step. Formulations so prepared may then be filled into the receptacle.

Aqueous formulations of the invention may also be employed for rectal, aural, optic, oral, topical or parenteral administration or administration by inhalation for the treatment of other local inflammatory conditions (for example dermatitis, asthma, chronic obstructive pulmonary disease (COPD) and the like). For example formulations of the invention may be administered to the lung by nebulisation. Such formulations may employ excipients (for example preservatives, buffers and the like) appropriate for the route of administration.

Examples of disease states in which the formulation of the present invention has utility include inflammatory and/or allergic conditions of the nasal passages such as rhinitis for example seasonal and perennial rhinitis as well as other local inflammatory conditions such as asthma, COPD and dermatitis.

It will be appreciated by those skilled in the art that reference herein to treatment extends to prophylaxis as well as the treatment of established conditions.

As mentioned above, formulations of the present invention are useful in human or veterinary medicine, in particular as an anti-inflammatory and anti-allergic agent.

There is thus provided as a further aspect of the invention a pharmaceutical formulation comprising a compound of formula (I) or a salt or solvate thereof and a corticosteroid selected from the group consisting of a compound of formula (II), a compound of formula (III), a compound of formula (IV), a compound of formula (V) and a solvate thereof, for use in human or veterinary medicine, particularly in the treatment of patients with an inflammatory and/or allergic condition.

According to another aspect of the invention, there is provided the use of a formulation comprising a compound of formula (I) or a salt or solvate thereof and a corticosteroid selected from the group consisting of a compound of formula (II), a compound of formula (III), a compound of formula (IV), a compound of formula (V) and a solvate thereof, for the manufacture of a medicament for the treatment of patients with an inflammatory and/or allergic condition.

In a further or alternative aspect, there is provided a method for the treatment of a human or animal subject with an inflammatory and/or allergic condition, which method comprises administering to said human or animal subject an effective amount of a formulation comprising a compound of formula (I) or a salt or solvate thereof and a corticosteroid selected from the group consisting of a compound of formula (II), a compound of formula (III), a compound of formula (IV), a compound of formula (V) and a solvate thereof.

In an alternative aspect there is provided a method of treatment of allergic rhinitis which comprises administering to a patient a pharmaceutically acceptable amount of a pharmaceutical formulation according to the present invention. In one embodiment administration is once-per-day.

The formulations of the present invention may be long-acting, therefore the formulation may be administered once daily and the dose may be selected so that the compounds have a therapeutic effect in the treatment of respiratory disorders (for example rhinitis) over 24 hours or more.

Processes for preparing compounds (II), (III), (IV) and (V) are known and are disclosed in WO02/12265, WO02/088167, WO05/005452 and WO02/12266 respectively. Processes for preparing a compound of formula (I) are also known and are disclosed in, for example, U.S. Pat. No. 3,813,384.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The patents and patent applications described in this application are herein incorporated by reference.

### BRIEF DESCRIPTION OF THE SEVERAL VIEWS OF THE DRAWING

FIG. 1 is a flow diagram illustrating a method of preparing the formulation of Example 1.

### DETAILED DESCRIPTION

The following non-limiting Examples illustrate the invention:

### EXAMPLES

### Example 1

### Nasal formulation containing 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and azelastine hydrochloride

A formulation for intranasal delivery may be prepared with ingredients as follows:

| | | Quantity (g per |
|---|---|---|
| Ingredients | Quantity (% w/w) | 50 L/spray) |
| 6α,9α-difluoro-11β-hydroxy-16α- | 0.05 | 25 |
| methyl-3-oxo-17α-(2,2,3,3- | | |
| tetramethycyclopropylcarbonyl)oxy- | | |
| androsta-1,4-diene-17β-carbothioic | | |
| acid S-cyanomethyl ester. | | |
| Azelastine Hydrochloride | 0.28 | 140 |
| Glucose Anhydrous | 5 | 2500 |
| Dispersible cellulose | 1.5 | 750 |
| Polysorbate 80 | 0.005 | 2.5 |
| Benzalkonium Chloride Solution | 0.03 | 15 |
| Disodium Edetate | 0.015 | 7.5 |
| Purified Water | to 100 | qs |

Hydrochloric acid or sodium hydroxide may be added to adjust the pH to 5.5-6.5, if required.

### Example 2

### Method of Preparing the Formulation of Example 1

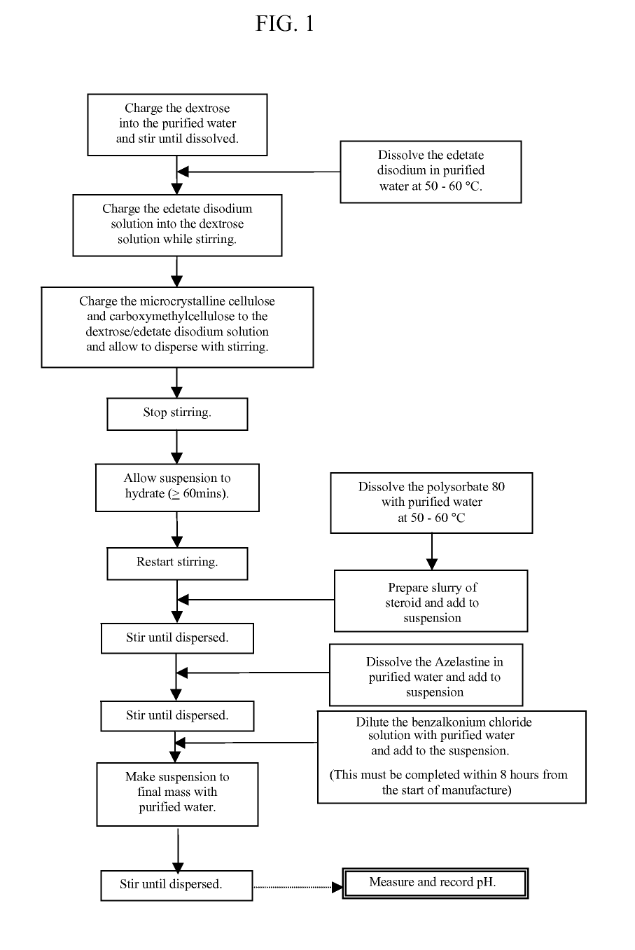

The formulation may be prepared by following the flow diagram in FIG. 1.

What is claimed is:

1. A method for the treatment of allergic rhinitis comprising administrating to a patient in need thereof a pharmaceutical formulation comprising: a compound of formula (I) 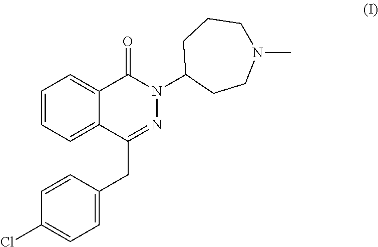
or a salt thereof, and a corticosteroid compound of formula (II) 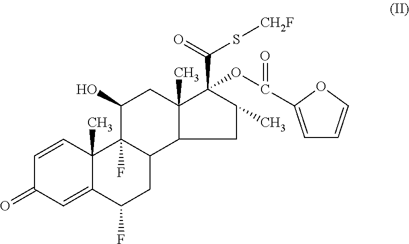
or a solvate of said corticosteroid,
wherein said pharmaceutical formulation is administered once-daily, and
wherein the compound of formula (I) or a salt thereof is present within the formulation in an amount from 0.0005% to 2% (w/w), based on the total weight of the formulation, and
wherein the corticosteroid compound of formula (II) or a solvate thereof is present within the formulation in an amount from 0.005% to 1% (w/w), based on the total weight of the formulation.

2. The method according to claim 1, wherein the compound of formula (I) is azelastine either in racemic form or a single enantiomer thereof.

3. The method according to claim 2, wherein the azelastine is present as the hydrochloride salt.

4. The method according to claim 1, wherein said pharmaceutical formulation further comprises one or more suspending agents.

5. The method according to claim 1, wherein said pharmaceutical formulation comprises one or more preservatives.

6. The method according to claim 1, wherein said pharmaceutical formulation comprises one or more wetting agents.

7. The method according to claim 1, wherein said pharmaceutical formulation comprises one or more isotonicity adjusting agents.

8. The method according to claim 1, wherein said pharmaceutical formulation comprises a buffer.

9. The method according to claim 1, wherein said pharmaceutical formulation comprises one or more taste-masking agents.

10. The method according to claim 1, wherein said pharmaceutical formulation comprises
(i) an aqueous solution of a compound of formula (I) or a salt thereof;
(ii) an aqueous suspension of a corticosteroid of formula (II), or a solvate thereof;
(iii) one or more suspending agents;
(iv) one or more preservatives;
(v) one or more wetting agents;
(vi) a buffer;
(vii) one or more isotonicity adjusting agents; and optionally
(viii) one or more taste-masking agents.
